SEQUENCE LISTING (6 sequences)

SEQ ID NO: 1 - DNA, 1392 bp, chicken embryos
atggctttgttctacacgggtttttttccctctacctgcaggctggagctacggtggtctcgcattgaaatgcagcagaccgaatatgaagtgtgtgagaacgtggggacggtgtccctgaaaatcaccagggcaggacactctgctgactctgcttttatcgcagtgaaggtcaatgaaatatctgctgtgctgggaaaggactttacagtgactccctccaaacttattcagtttgatccaggaatgtcaaccaagatgtggaatatagcaattacttatgacagattagaggaagatgatgaagtctttgaagtagttctgaactcctctgtgaatgctgttcttggcaccaagacaaaagctgtagtgaaaattctggactcaaaaggaggtcagtgcagtactttacattcttccagccaaagcaagcaagacttctggaggagaggcacactgtttgcagtttcctcaggctcctcatcacctcccaggcctggcaatgttcacttggaagggatctcaccacctccttccaaaaggatgagagaacacagaggtgacacactgcaggaccgcaggttggcgtatcagtcaagggccaaactgagagtgactggaaatggcagaatagttcatccttcatctatatttagaaatggaacagatgtggtttacaaataccatgggatagtatcactcaaaatagaggatgacacctcatcagctaaagcaaacaagaaggctgaagtgtcagtaattaaccaagcacaaccacagataaatgccatttccactaggaagacagaaatcccacaagctgataaggcagaactggcatctgaagcaagctcaagacgtcaggactttcactccttttcaagggcctgtacaccagatttgaaggggctcatgcattatgaagaaagcactcagaagttatttcagtgtgacgggatagaatggaaattttggagttcccaaagcaaggaggcaagcatgaaaaaatgtccctctggatggcgtcatcatgatggcagttgctatttcctggtagtgaatcaaaaggtcacctgggatactgctgctcaggcctgcagagaacaatacctggggagtctcacaagtgtggctaatgaacaacacatgcagtggctgtgggacttaggcgggaggaagcccttttggataggcttgaatgaccaagtcaatcctggacactgggagtggaacggaggagaacctgttacctacataaactggagaagaggctcctatcgctttacaaagagaggcaacaactgtgtagtggtgcagaagagaggaaaatggcaagcaactgactgcaggaaggccaaatggaacagctacatatgttcaagaaagctgtaa

SEQ ID NO: 2 - protein, 463 aa, chicken embryos
MALFYTGFFPSTCRLELRWSRIEMQQTEYEVCENVGTVSLKITRAGHSADSAFIAVKVNEISAVLGKDFTVTPSKLIQFDPGMSTKMWNIAITYDRLEEDDEVFEVVLNSSVNAVLGTKTKAVVKILDSKGGQCSTLHSSSQSKQDFWRRGTLFAVSSGSSSPPRPGNVHLEGISPPPSKRMREHRGDTLQDRRLAYQSRAKLRVTGNGRIVHPSSIFRNGTDVVYKYHGIVSLKIEDDTSSAKANKKAEVSVINQAQPQINAISTRKTEIPQADKAELASEASSRRQDFHSFSRACTPDLKGLMHYEESTQKLFQCDGIEWKFWSSQSKEASMKKCPSGWRHHDGSCYFLVVNQKVTWDTAAQACREQYLGSLTSVANEQHMQWLWDLGGRKPFWIGLNDQVNPGHWEWNGGEPVTYINWRRGSYRFTKRGNNCVVVQKRGKWQATDCRKAKWNSYICSRKL

SEQ ID NO: 3 - DNA, 387 bp, chicken embryos
aaaaaatgtccctctggatggcgtcatcatgatggcagttgctatttcctggtagtgaatcaaaaggtcacctgggatactgctgctcaggcctgcagagaacaatacctggggagtctcacaagtgtggctaatgaacaacacatgcagtggctgtgggacttaggcgggaggaagcccttttggataggcttgaatgaccaagtcaatcctggacactgggagtggaacggaggagaacctgttacctacataaactggagaagaggctcctatcgctttacaaagagaggcaacaactgtgtagtggtgcagaagagaggaaaatggcaagcaactgactgcaggaaggccaaatggaacagctacatatgttcaagaaagctg

SEQ ID NO: 4 - protein, 129 aa, chicken embryos
KKCPSGWRHHDGSCYFLVVNQKVTWDTAAQACREQYLGSLTSVANEQHMQWLWDLGGRKPFWIGLNDQVNPGHWEWNGGEPVTYINWRRGSYRFTKRGNNCVVVQKRGKWQATDCRKAKWNSYICSRKL

SEQ ID NO: 5 - protein, 410 aa, Artificial Sequence
  Curagen DP214
MDSAFVGIKVNQVSAAVGKDFTVIPSKLIQFDPGMSTKMWNIAITYDGLEEDDEVFEVILNSPVNAVLGTKTKAAVKILDSKGGQCHPSYSSNQSKHSTWEKGIWHLLPPGSSSSTTSGSFHLERRPLPSSMQLAVIRGDTLRGFDSTDLSQRKLRTRGNGKTVRPSSVYRNGTDIIYNYHGIVSLKLEDDSFPTHKRKAKVSIISQPQKTIKVAELPQADKVESTTDSHFPRQDQLPSFPKNCTLELKGLFHFEEGIQKLYQCNGIAWKAWSPQTKDVEDKSCPAGWHQHSGYCHILITEQKGTWNAAAQACREQYLGNLVTVFSRQHMRWLWDIGGRKSFWIGLNDQVHAGHWEWIGGEPVAFTNGRRGPSPRSKLGKSCVLVQRQGKWQTKDCRRAKPHNYVCSRKL

SEQ ID NO: 6 - protein, 456 aa, Artificial Sequence
  Incyte DP214
MDPTGNSATPQILELKWSHIEWSQTEYICENVGLLPLEIIRRGYSMDSAFVGIKVNQVSAAVGKDFTVIPSKLIQFDPGMSTKMWNIAITYDGLEEDDEVFEVILNSPVNAVLGTKTKAAVKILDSKGGQCHPSYSSNQSKHSTWEKIGIWHLLPPGSSSSTTSGSFHLERRPLPSSMQLAVIRGDTLRGFDSTDLSQRKLRTRGNGKTVRPSSVYRNGTDIIYNYHGIVSLKLEDDSFPTHKRKAKVSIISQPQKTIKVAELPQADKVESTTDSHFPRQDQLPSFPKNCTLELKGLFHFEEGIQKLYQCNGIAWKAWSPQTKDVEDKSCPAGWHQHSGYCHILITEQKGTWNAAAQACREQYLGNLVTVFSRQHMRWLWDIGGRKSFWIGLNDQVHAGHWEWIGGEPVAFTNGRRGPSPRSKLGKSCVLVQRQGKWQTKDCRRAKPHNYVCSRKL

(19) United States
(12) Patent Application Publication
(10) Pub. No.: US 2004/0072773 A1
(43) Pub. Date: Apr. 15, 2004

(54) Novel functions for dp214

(76) Inventor: Cord E. Dohrmann, Gottingen (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/363,426

(22) PCT Filed: Aug. 31, 2001
(86) PCT No.: PCT/EP01/10076

(30) Foreign Application Priority Data
Sep. 1, 2000 (DE) 100 43 227.1

(51) Int. Cl. A61K 48/00

(52) U.S. Cl. 514/44

(57) ABSTRACT

The present invention discloses polynucleotides which identify and encode pDP214 as well as novel functions for the DP214 gene family (pDP214) which are specifically expressed specifically expressed in the mesenchyme of the developing pancreas, in the lung, the stomach, the kidney and specific areas of the ectoderm such as ectodermal ridge and hair follicles. The invention also provides genetically engineered expression vectors and host cells comprising the nucleic acid sequences encoding pDP214 and a method for producing pDP214. The invention also provide for agonists, antibodies, or antagonists specifically binding, pDP214, and their, use, in the prevention and treatment of diseases associated with the expression of pDP214. Additionally, the invention provides for the use or antisense molecules to polynucleotides encoding pDP214 for the treatment or diseases associated with the expression of pDP214. The invention also provides diagnostic assays which utilize the polynucleotide, or fragments or the complement thereof, and antibodies specifically binding pDP214.

### FIELD OF THE INVENTION

This invention relates to nucleic acid and amino acid sequences of DP214-like proteins specifically expressed in the mesenchyme of the developing pancreas, the lung, the stomach, the kidney, and specific areas of the ectoderm such as ectodermal ridge and hair follicles, and to the use of these sequences in the diagnosis, study, prevention, and treatment of diseases and disorders, for example, but not limited to, of the pancreas (such as diabetes), and related diseases and disorders (such as adipositas), of the lung, of the stomach, of the kidney, of the skin, of appendages such as limbs and hair, and others.

### BACKGROUND OF THE INVENTION

The protein described in this invention shows significant homologies to a hypothetical human protein (for example, GenBank Accession Number AAC66913 or GenBank Accession Number AAC76594). A function of this human protein in the regulation of diseases and disorders, particularly of the pancreas (such as diabetes), and related diseases and disorders (such as adipositas), of the lung, of the stomach, of the kidney, and others has not been described in the prior art. This invention describes novel functions for the DP214 gene family that is involved in the development of the pancreas, the lung, the stomach, the kidney, the skin and appendages such as limbs and hair.

The discovery of polynucleotides encoding molecules specifically expressed in the mesenchyme of the developing pancreas, in the lung, the stomach, in the kidney, and specific areas of the ectoderm such as ectodermal ridge and hair follicles presents the opportunity to investigate diseases and disorders of the pancreas including diabetes and obesity. Furthermore, it presents the opportunity to investigate diseases and disorders affecting the lung, the gastrointestinal tract, the uro-genital tragt as well as developing appendages like limbs and hair. Discovery of molecules related to the DP214 protein satisfies a need in the art by providing new compositions useful in diagnosis, prevention, treatment, and prognosis of diseases and disorders, for example, but not limited to, of the pancreas (such as diabetes), and related diseases and disorders (such as adipositas), of the lung, of the stomach, of the kidney, of the skin and of appendages and others.

### SUMMARY OF THE INVENTION

The present invention features a DP214-like protein specifically expressed in the mesenchyme of the developing pancreas, in the lung, the stomach, the kidney, and specific areas of the ectoderm such as ectodermal ridge and hair follicles. The protein of the invention has been further characterized as having similarity to a hypothetical human protein (for example, GenBank Accession Number AAC66913, GenBank Accession Number AAC76594 and WO00/68380 from Incyte or WO00/58473 from Curagen).

Accordingly, the invention relates to isolated and substantially purified nucleic acid molecules including a differentiation gene or a developmental control gene comprising the nucleotide sequence shown in SEQ ID NO:1, a derivative or fragment thereof or a sequence hybridizing thereto or to its complement. Preferably the nucleic acid molecule comprises a sequence associated with the pancreas development. In addition to the sequence of SEQ ID NO:1 which is derived from chicken or its human homolog, the invention particularly encompasses modified sequences having the same or a similar control function as the sequence of SEQ ID NO:1.

A further aspect of the invention features isolated and substantially-purified polynucleotides that encode a DP214 protein (pDP214). In a particular aspect, the polynucleotide is the nucleotide sequence of SEQ ID NO:1. The invention also relates to a polynucleotide sequence comprising the complement of SEQ ID NO:1 or variants thereof. In addition, the invention features polynucleotide sequences which hybridize under stringent conditions to SEQ ID NO:1. The invention additionally features nucleic acid sequences including oligonucleotides, peptide nucleic acids (PNA), locked nucleic acids (LNA), morpholino nucleic acids, fragments, portions or antisense molecules thereof, and expression vectors and host cells comprising polynucleotides that encode pDP214 or fragments thereof, particularly fragments suitable for diagnostic and/or therapeutic applications.

Further, the invention features any polypeptide or peptide encoded by nucleic acid molecule as described above. Particularly, the invention relates to a substantially purified pDP214 which has the amino acid sequence shown in SEQ ID NO:2.

The present invention also features antibodies and aptamers which bind specifically to pDP214. The invention also features the identification and the use of agonists and antagonists of pDP214.

Finally, the invention relates to pharmaceutical compositions comprising DP214 nucleic acids, proteins, antibodies and/or aptamers as active ingredient.

### BRIEF DESCRIPTION OF THE FIGURES

FIG. 1: Nucleic acid sequence (SEQ ID NO:1) encoding the DP214-like protein of chicken DP214.

FIG. 2: Protein sequence (SEQ ID NO:2) encoded by the coding sequence shown in FIG. 1.

FIG. 3A: Nucleic acid sequence (SEQ ID NO:3) encoding the C-type lectin domain of the DP214-like protein of chicken (nucleotides 1778 to 2164, 387 base pairs).

FIG. 3B: Amino acid sequence(SEQ ID NO:4) of the C-type lectin domain of the DP214-like protein of chicken (129 amino acids).

FIG. 4: In situ hybridization results for the DP214-like protein of the invention.

FIG. 4A shows whole-mount in situ hybridizatons on chick embryos.

FIG. 4B shows the dorsal view of myotomes of the same chick embryos (day 5).

FIG. 4C and FIG. 4D show in situ hybridizations on pancreatic bud tissue sections (5 day old embryos)

FIG. 4E shows in situ hybridizations on sagittal sections (5 day old embryos) detecting the expression of DP214 mRNA in the mesenchyml dorsal pancreatic buds (DP214 shown in blue, Insulin shown in brown color).

FIG. 5 shows a comparison between chick DP214 protein (FIG. 1) and human DP214 homologs (‘Curagen DP214’ and ‘Incyte DP214’).

### DESCRIPTION OF THE INVENTION

Before the present proteins, nucleotide sequences, and methods are described, it is understood that this invention is not limited to the particular methodology, protocols, cell lines, vectors, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies, and equivalents thereof known to those skilled in the art, and so forth. Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies which are reported in the publications which might be used in connection with the invention. Nothing herein is to be construed as an admission that the invention is not entitled to antedate such disclosure by virtue of prior invention.

### THE INVENTION

The invention is based on the discovery of novel functions for DP214-like proteins and nucleic acids coding therefor. It is described in this invention that pDP214 protein is specifically expressed in the mesenchyme of the developing pancreas, in the lung, the stomach, the kidney and specific areas of the ectoderm such as ectodermal ridge and hair follicles. The invention is based on the use of polynucleotides encoding pDP214 for the diagnosis, study, prevention, or treatment of diseases and disorders related to such cells, including diseases and disorders, for example, but not limited to, of the pancreas (such as diabetes), and related diseases and disorders (such as adipositas), of the lung, of the stomach, of the kidney, of the skin, of appendages and others, particularly in mammals.

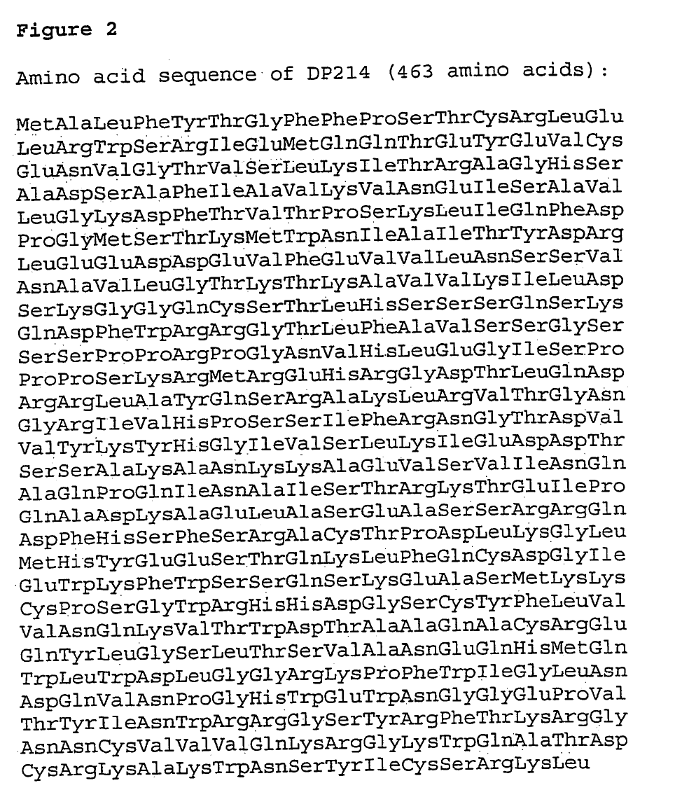

Nucleic acids encoding the chicken pDP214 of the present invention were first identified in DeveloGen Clone DP214 from the pancreas tissue cDNA library (day 6) through a whole-mount in situ screen for genes expressed in the embryonic pancreatic bud. In one embodiment, the invention encompasses the DP214-like protein specifically in the mesenchyme of the developing pancreas, in the lung, the stomach, the kidney and specific areas of the ectoderm such as ectodermal ridge and hair follicles, a polypeptide comprising the amino acid sequence of SEQ ID NO:2, as presented using the one-letter code in FIG. 2. pDP214 is 463 amino acids in length. An open reading was identified beginning with an ATP initiation codon at nucleotide 776 and ending with a TGA stop codon at nucleotide 2167 (shown in FIG. 2). The calculated molecular weight of the protein of the invention is 52397 daltons.

The predicted amino acid sequence was searched in the publicly available GenBank database. In search of sequence databases, it was found, for example, that pDP214 has homology with a hypothetical human protein (Genbank Accession Number AAC66913 and Genbank Accession Number AAC76594). Based upon homology, pDP214 protein and each homologous protein or peptide have at least similar activities.

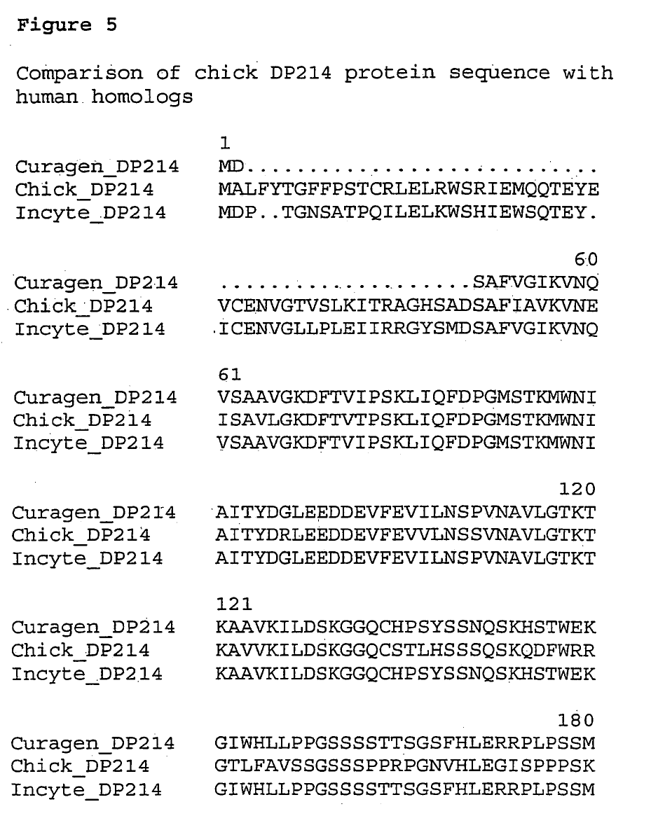

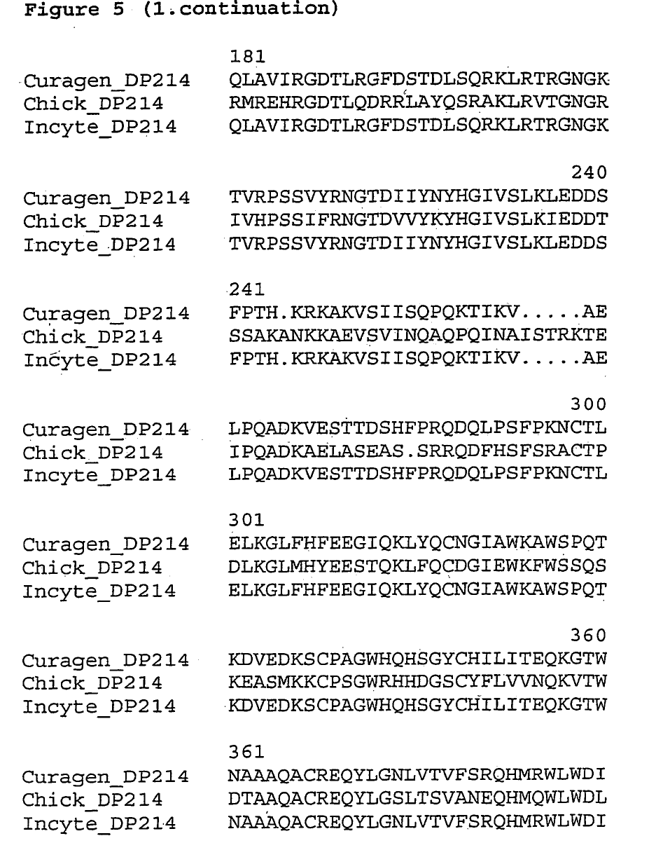

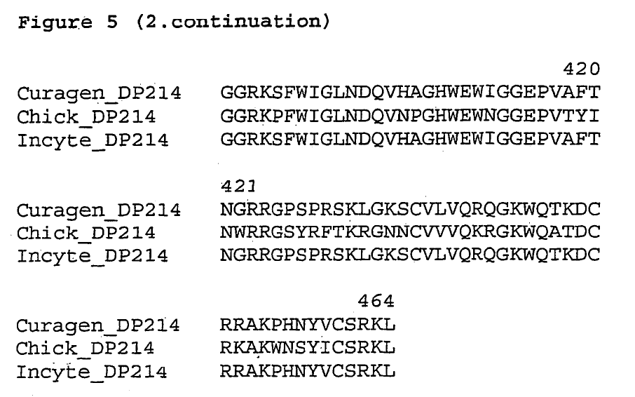

A multiple sequence aligment is given in FIG. 5 with the protein of the invention being shown on line 1, in a ClustalX analysis comparing the protein of the invention (line 1, ‘Chick DP214’) with a related human protein sequence (line 2, ‘Curagen DP214, line 3, ‘Incyte DP214’).

Expression

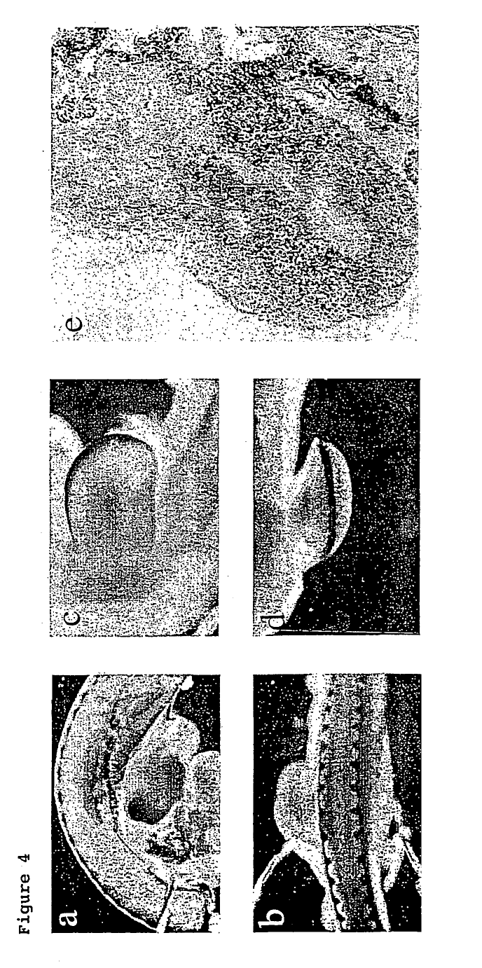

In situ hybridization experiments using the antisense-RNA probes complemtary to DP214 mRNAs were carried out on whole mounts of 4 (FIG. 4A) and 5-day-old chick embryos (FIGS. 4B and C) and on sectioned pancreatic bud tissue (FIGS. 4D and E) and sections neural tissue. The hybridizations show that transcripts of the invention are exclusively expressed in the mesenchyme of the developing pancreas, in the lung, the stomach, the kidney and specific areas of the ectoderm such as ectodermal ridge and hair follicles (arrows, FIGS. 4D and E).

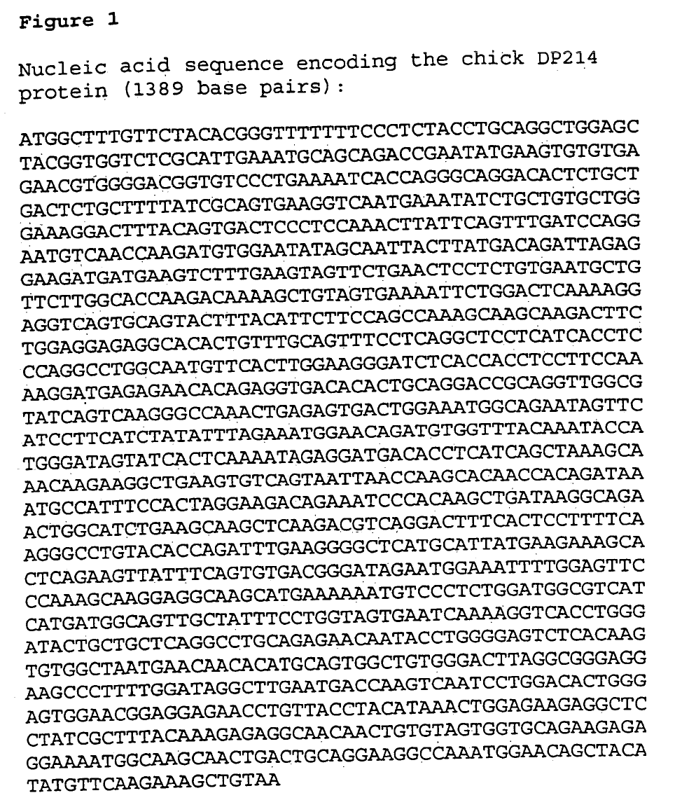

The invention also encompasses polynucleotides which encode pDP214. Accordingly, any nucleic acid sequence which encodes the amino acid sequence of pDP214 can be used to generate recombinant molecules which express pDP214. In a particular embodiment, the invention encompasses the polynucleotide comprising the nucleic acid sequence of SEQ ID NO:1 as shown in FIG. 1A.

It will be appreciated by those skilled in the art that as a result of the degeneracy of the genetic code, of nucleotide sequences encoding pDP214, different from the nucleotide sequences of any known and naturally occurring gene, may be produced. Thus, the invention contemplates each and every possible variation of nucleotide sequence that could be made by selecting combinations based on possible codon choices. These combinations are made in accordance with the standard triplet genetic code as applied to the nucleotide sequence of naturally occurring pDP214, and all such variations are to be considered as being specifically disclosed. Although nucleotide sequences which encode pDP214 and its variants are preferably capable of hybridizing to the nucleotide sequence of the naturally occurring pDP214 under appropriately selected conditions of stringency, it may be advantageous to produce nucleotide sequences encoding pDP214 or its derivatives possessing a substantially different codon usage. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding pDP214 and its derivatives without altering the encoded amino acid sequences include the production of RNA transcripts having more desirable properties, such as a greater half-life, than transcripts produced from the naturally occurring sequence.

The invention also encompasses production of DNA sequences, or portions therof, which encode pDP214 and its derivatives, entirely by synthetic chemistry. After production, the synthetic sequence may be inserted into any of the many available expression vectors and cell systems using reagents that are well known in the art at the time of the filing of this application. Moreover, synthetic chemistry may be used to introduce mutations into a sequence encoding pDP214 or any portion thereof.

Also encompassed by the invention are polynucleotide sequences that are capable of hybridizing to the claimed nucleotide sequences, and in particular, those shown in SEQ ID NO:2, under various conditions of stringency. Hybridization conditions are based on the melting temperature (Tm) of the nucleic acid binding complex or probe, as taught in Wahl, G. M. and S. L. Berger (1987: Methods Enzymol. 152:399-407) and Kimmel, A. R. (1987; Methods Enzymol. 152:507-511), and may be used at a defined stringency. Preferably, a hybridization under stringent conditions means that a positive hybridization signal is observed after washing for 1 hour with 1×SSC buffer and 0.1% SDS at 55° C., preferably at 62° C. and most preferably at 68° C., in particular, for 1 h in 0.2×SSC buffer and 0.1% SDS at 55° C., preferably at 62° C. and most preferably at 68° C.

Altered nucleic acid sequences encoding pDP214 which are encompassed by the invention include deletions, insertions, or substitutions of different nucleotides resulting in a polynucleotide that encodes the same or a functionally equivalent pDP214. The encoded protein may also contain deletions, insertions, or substitutions of amino acid residues which produce a silent change and result in a functionally equivalent pDP214. Deliberate amino acid substitutions may be made on the basis of similarity in polarity, charge, solubility, hydrophobicity, hydrophilicity, and/or the amphipathic nature of the residues as long as a biological activity of pDP214 is retained. For example, negatively charged amino acids may include aspartic acid and glutamic acid; positively charged amino acids may include lysine and arginine; and amino acids with uncharged polar head groups having similar hydrophilicity values may include leucine, isoleucine, and valine; glycine and alanine; asparagine and glutamine; serine and threonine; phenylalanine and tyrosine.

Also included within the scope of the present invention are alleles of the genes encoding pDP214. As used herein, an “allele” or “allelic sequence” is an alternative form of the gene which may result from at least one mutation in the nucleic acid sequence. Alleles may result in altered mRNAs or polypeptides whose structures or function may or may not be altered. Any given gene may have none, one, or many allelic forms. Common mutational changes which give rise to alleles are generally ascribed to natural deletions, additions, or substitutions of nucleotides. Each of these types of changes may occur alone, or in combination with the others, one or more times in a given sequence.

Methods for DNA sequencing which are well known and generally available in the art may be used to practice any embodiments of the invention. The nucleic acid sequences encoding pDP214 may be extended utilizing a partial nucleotide sequence and employing various methods known in the art to detect upstream sequences such as promoters and regulatory elements. For example, one method which may be employed, “restriction-site” PCR, uses universal primers to retrieve unknown sequence adjacent to a known locus (Sarkar, G. (1993) PCR Methods Applic. 2:318-322). In particular, genomic DNA is first amplified in the presence of primer to linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase. Inverse PCR may also be used to amplify or extend sequences using divergent primers based on a known region (Triglia, T. et al. (1988) Nucleic Acids Res. 16:81.86). The primers may be designed using OLIGO 4.06 primer analysis software (National Biosciences Inc., Plymouth, Minn.), or another appropriate program, to 22-30 nucleotides in length, to have a GC content of 50% or more, and to anneal to the target sequence temperatures about 68° C.-72° C. The method uses several restriction enzymes to generates suitable fragment. The fragment is then circularized by intramolecular ligation and used as a PCR template.

Another method which may be used is capture PCR which involves PCR amplification of DNA fragments adjacent to a known sequence in human and yeast artificial chromosome DNA (Lagerstrom, M. et al. (PCR Methods Applic. 1:111-119). In this method, multiple restriction enzyme digestions and ligations also be used to place an engineered double-stranded sequence into an unknown portion of the DNA molecule before performing PCR. Another method which may be used to retrieve unknown sequences is that of Parker, J. D. et al. (1991; Nucleic Acids Res. 19:3055-3060). Additionally, one may use PCR, nested primers, and PROMOTERFINDER libraries to walk in genomic DNA (Clontech, Palo Alto, Calif.). This process avoids the need to screen libraries and is useful in finding intron/exon junctions. When screening for full-length cDNAs, it is preferable to use libraries that have been size-selected to include larger cDNAs. Also, random-primed libraries are preferable, in that they will contain more sequences which contain the 5′ regions of genes. Use of a randomly primed library may be especially preferable for situations in which an oligo d(T) library does not yield a full-length cDNA. Genomic libraries may be useful for extension of sequence into the 5′ and 3′ non-transcribed regulatory regions. Capillary electrophoresis systems which are commercially available may be used to analyze the size or confirm the nucleotide sequence of sequencing or PCR products. In particular, capillary sequencing may employ flowable polymers for electrophoretic separation, four different fluorescent dyes (one for each nucleotide) which are laser activated, and detection of the emitted wavelengths by a charge coupled devise camera. An output/light intensity may be converted to electrical signal using appropriate software (e.g. GENOTYPER and SEQUENCE NAVIGATOR, Perkin Elmer) and the entire process from loading of samples to computer analysis and electronic data display may be computer controlled. Capillary electrophoresis is especially preferable for the sequencing of small pieces of DNA which might be present in limited amounts in a particular sample.

In another embodiment of the invention, polynucleotide sequences or fragments thereof which encode pDP214, or fusion proteins or functional equivalents thereof, may be used in recombinant DNA molecules to direct expression of pDP214 in appropriate host cells. Due to the inherent degeneracy of the genetic code, other DNA sequences which encode substantially the same or a functionally equivalent amino acid sequence may be produced and these sequences may be used to clone and expres pDP214. As will be understood by those of skill in the art, it may be advantageous to produce pDP214-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence. The nucleotide sequences of the present invention can be engineered using methods generally known in the artm in order to alter pDP214 encoding sequences for a variety of reasons, including but not limited to, alterations, which modify the cloning, processing, and/or expression of the gene product. DNA shuffling by random fragmentation and PCR reassembly of gene fragments and synthetic oligonucleotides may be used to engineer the nucleotide sequences. For example, site-directed mutagenesis may be used to insert new restriction sites, alter glycosylation patterns, change codon preference, produce splice variants, or introduce mutations, and so forth. In another embodiment of the invention, natural, modified, or recombinant nucleic acid sequences encoding pDP214 may be ligated to a heterologous sequence to encode a fusion protein. For example, to screen peptide libraries for inhibitors of pDP214 activity, it may be useful to encode a chimeric pDP214 protein that can be recognized by a commercially available antibody. A fusion protein may also be engineered to contain a cleavage site located between the pDP214 encoding sequence and the heterologous protein sequence, so that pDP214 may be cleaved and purified away from the heterologous moiety. In another embodiment, sequences encoding pDP214 may be synthesized, in whole or in part, using chemical methods well known in the art (see Caruthers, M. H. et al. (1980) Nucl. Acids Res. Symp. Ser. 7:215-223, Horn, T. et al. (1980) Nucl. Acids Res. Symp. Ser. 7:225-232).

Alternatively, the protein itself may be produced using chemical methods to synthesize the amino acid sequence of pDP214, or a portion thereof. For example, peptide synthesis can be performed using various solid-phase techniques (Roberge, J. Y. et al. (1995) Science 269:202-204) and automated synthesis may be achieved, for example, using the ABI 431A peptide synthesizer (Perkin Elmer). The newly synthesized peptide may be substantially purified by preparative high performance liquid chromatography (e.g., Creighton, T. (1983) Proteins, Structures and Molecular Principles, W H Freeman and Co., New York, N.Y.). The composition of the synthetic peptides may be confirmed by amino acid analysis or sequencing (e.g., the Edman degradation procedure; Creighton, supra). Additionally, the amino acid sequence of pDP214, or any part thereof, may be altered during direct synthesis and/or combined using chemical methods with sequences from other proteins, or any part thereof, to produce a variant polypeptide.

In order to express a biologically active pDP214, the nucleotide sequences encoding pDP214 or functional equivalents, may be inserted into appropriate expression vector, i.e., a vector which contains the necessary elements for the transcription and translation of the inserted coding sequence. Methods which are well known to those skilled in the art may be used to construct expression vectors containing sequences encoding pDP214 and appropriate transcriptional and translational control elements. These methods include in vitro recombinant DNA techniques, synthetic techniques, and in vivo genetic recombination. Such techniques are described in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y. A variety of expression vector/host systems may be utilized to contain and express sequences encoding pDP214. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or PBR322 plasmids); or animal cell systems. The “control elements” or “regulatory sequences” are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the BLUESCRIPT phagemid (Stratagene, LaJolla, Calif.) or PSPORT1 plasmid (Gibco BRL) and the like may be used. The baculovirus polyhedrin promoter may be used in insect cells. Promoters and enhancers derived from the genomes of plant cells (e.g., heat shock, RUBISCO; and storage protein genes) or from plant viruses (e.g., viral promoters and leader sequences) may be cloned into the vector. In mammalian cell systems, promoters from mammalian genes or from mammalian viruses are preferable. If it is necessary to generate a cell line that contains multiple copies of the sequence encoding pDP214, vectors based on SV40 or EBV may be used with an appropriate selectable marker.

In bacterial systems, a number of expression vectors may be selected depending upon the use intended for pDP214. For example, when large quantities of pDP214 are needed for the induction of antibodies, vectors which direct high level expression of fusion proteins that are readily purified may be used. Such vectors include, but are not limited to, the multifunctional E. coli. cloning and expression vectors such as the BLUESCRIPT phagemid (Stratagene), in which the sequence encoding pDP214 may be ligated into the vector in frame with sequences for the amino-terminal Met and the subsequent 7 residues of β-galactosidase so that a hybrid protein is produced; pIN vectors (Van Heeke, G. and S. M. Schuster (1989). J. Biol. Chem. 264:5503-5509); and the like. PGEX vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In yeast, e.g. in Saccharomyces cerevisiae, a number of vectors containing constitutive or inducible promoters such as alpha factor, alcohol oxidase, and PGH may be used. For reviews, see Ausubel et al., (supra) and Grantet al. (1987) Methods Enzymol. 153:516-544.

In cases where plant expression vectors are used, the expression of sequences encoding pDP214 may be driven by any of a number of promoters. For example, viral promoters such as the 35S and 19S promoters of CaMV may be used alone or in combination with the omega leader sequence from TMV (Takamatsu, N. (1987) EMBO J. 6:307-311). Alternatively, plant promoters such as the small subunit of RUBISCO or heat shock promoters may be used (Coruzzi, G. et al. (1984) EMBO J. 3:1671-1680; Broglie, R. et al. (1984) Science 224:838-843; and Winter, J. et al. (1991) Results Probl. Cell Differ. 17:85-105). These constructs can be introduced into plant cells by direct DNA transformation or pathogen-mediated transfection. Such techniques are described in a number of generally available reviews (see, for example, Hobbs, S. or Murry, L. E. in McGraw Hill Yearbook of Science and Technology (1992) McGraw Hill, New York, N.Y.; pp. 191-196.

An insect system may also be used to express pDP214. For example, in, one such system, Autographa californica nuclear polyhedrosis virus (AcNPV) is used as a vector to express foreign genes in Spodoptera frugiperda cells or in Trichoplusia larvae. The sequences encoding pDP214 may be cloned into a non-essential region of the virus, such as the polyhedrin gene, and place under control of the polyhedrin promoter. Successful insertion of pDP214 will render the polyhedrin gene inactive and produce recombinant virus lacking coat protein. The recombinant viruses may then be used to infect, for example, S. frugiperda cells of Trichoplusia larvae in which pDP214 maybe expressed (Engelhard, E. K. et al. (1994) Proc. Nat. Acad. Sci. 91:3224-3227).

In mammalian host cells, a number of viral-based expression systems may be utilized. In cases where an adenovirus is used as an expression vector, sequences encoding pDP214 may be ligated into an adenovirus transcription/translation complex consisting of the late promoter and tripartite leader sequence. Insertion in a non-essential E1 or E3 region of the viral genome may be used to obtain a viable virus which is capable of expressing pDP214 in infected host cells (Logan, J. and Shenk, T. (1984) Proc. Natl. Acad. Sci. 81:3655-3659). In addition, transcription enhancers, such as the Rous sarcoma virus (RSV) enhancer, may be used to increase expression in mammalian host cells. Specific initiation signals may also be used to achieve more efficient translation of sequences encoding pDP214. Such signals include the ATG initiation codon and adjacent sequences.

In cases where sequences encoding pDP214, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf, D. et al. (1994) Results Probl. Cell Differ. 20:125-162). In addition, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells such as CHO, HeLa, MDCK, HEK293, and WI38, which have specific cellular machinery and characteristic mechanisms for such post-translational activities, may be chosen to ensure the correct modification and processing of the foreign protein.

For long-term, high-yield production of recombinant proteins, stable expression is preferred. For example, cell lines which stably express pDP214 may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type. Any number of selection systems may bemused to recover transformed cell lines. These include, but are not limited to, the herpes simplex virus thymidine kinase (Wigler, M. et al. (1977) Cell 11:223-32) and adenine phosphoribosyltransferase (Lowy, I. et al. (1980) Cell 22:817-23) genes which can be employed in tk- or aprt-cells, respectively. Also, antimetabolite, antibiotic or herbicide resistance can be used as the basis for selection; for example, dhfr which confers resistance to methotrexate (Wigler, M. et al. (1980) Proc. Natl. Acad. Sci. 77:3567-70); npt, which confers resistance to the aminoglycosides neomycin and G-418 (Colbere-Garapin, F. et al (1981) J. Mol. Biol. 150:1-14) and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman, S. C. and R. C. Mulligan (1988) Proc. Natl. Acad. Sci. 85:8047-51). Recently, the use of visible markers has gained popularity with such markers as anthocyanins, β glucuronidase and its substrate GUS, and luciferase and its substrate luciferin, being widely used not only to identify transformants, but also to quantify the amount of transient or stable protein expression attributable to a specific vector system (Rhodes, C. A. et al. (1995) Methods Mol. Biol. 55:121-131).

Although the presence/absence of marker gene expression suggests that the gene of interest is also present, its presence and expression may need to be confirmed. For example, if the sequence encoding pDP214 is inserted within a marker gene sequence, recombinant cells containing sequences encoding pDP214 can be identified by the absence of marker gene function. Alternatively, a marker gene can be placed in tandem with a sequence encoding pDP214 under the control of a single promoter. Expression of the marker gene in response to induction or selection usually indicates expression of the tandem gene as well.

Alternatively, host cells which contain the nucleic acid sequence encoding pDP214 and express pDP214 may be identified by a variety of procedures known to those of skill in the art. These procedures include, but are not limited to, DNA-DNA, or DNA-RNA hybridizations and protein bioassay or immunoassay techniques which include membrane, solution, or chip based technologies for the detection and/or quantification of nucleic acid or protein. The presence of polynucleotide sequences encoding pDP214 can be detected by DNA-DNA or DNA-RNA hybridization or amplification using probes or portions or fragments of polynucleotides encoding pDP214. Nucleic acid amplification based assays involve the use of oligonucleotides or oligomers based on the sequences encoding pDP214 to detect transformants containing DNA or RNA encoding pDP214. As used herein “oligonucleotides” or “oligomers” refer to a nucleic acid sequence of at least about 10 nucleotides and as many as about 60 nucleotides; preferably about 15 to 30 nucleotides, and more preferably about 20-25 nucleotides, which can be used as a probe or amplimer.

A variety of protocols for detecting and measuring the expression of pDP214, using either polyclonal or monoclonal antibodies specific for the protein are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on pDP214 is preferred, but a competitive binding assay may be employed. These and other assays are described, among other places, in Hampton, R. et al. (1990; Serological Methods, a Laboratory Manual, APS Press, St Paul, Minn.) and Maddox, D. E. et al. (1983; J. Exp. Med. 158:1211-1216).

A wide variety of labels and conjugation techniques are known by those skilled in the art and may be used in various nucleic acid and amino acid assays. Means for producing labeled hybridization or PCR probes for detecting sequences related to polynucleotides en coding pDP214 include oligolabeling, nick translation, end-labeling or PCR amplification using a labeled nucleotide. Alternatively, the sequences encoding pDP214, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits (Pharmacia & Upjohn, (Kalamazoo, Mich.); Promega (Madison Wis.); and U.S. Biochemical Corp., (Cleveland, Ohio). Suitable reporter molecules or labels, which may be used, include radionuclides, enzymes, fluorescent, chemiluminescent, or chromogenic agents as well as substrates, cofactors, inhibitors, magnetic particles, and the like.

Host cells transformed with nucleotide sequences encoding pDP214 may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides which encode pDP214 may be designed to contain signal sequences which direct secretion of pDP214 through a prokaryotic or eukaryotic cell membrane. Other recombinant constructions may be used to join sequences encoding pDP214 to nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAG extension/affinity purification system (Immunex Corp., Seattle, Wash.) The inclusion of cleavable linker sequences such as those specific for Factor XA or enterokinase (Invitrogen, San Diego, Calif.) between the purification domain and pDP214 may be used to facilitate purification. One such expression vector provides for expression of a fusion protein containing pDP214 and a nucleic acid encoding 6 histidine residues preceding a thioredoxin or an enterokinase cleavage site. The histidine residues facilitate purification on IMIAC (immobilized metal ion affinity chromotagraphy as described in Porath, J. et al. (1992, Prot. Exp. Purif. 3: 263-281) while the enterokinase cleavage site provides a means for purifying pDP214 from the fusion protein. A discussion of vectors which contain fusion proteins is provided in Kroll, D. J. et al. (1993; DNA Cell Biol. 12:441-453). In addition to recombinant production, fragments of pDP214 may be produced by direct peptide synthesis' using solid-phase techniques (Merrifield J. (1963) J. Am. Chem. Soc. 85:2149-2154). Protein synthesis may be performed using manual techniques or by automation. Automated synthesis may be achieved, for example, using Applied Biosystems 431A peptide synthesizer (Perkin Elmer). Various fragments of pDP214 may be chemically synthesized separately and combined using chemical methods to produce the full length molecule.

Diagnostics and Therapeutics

According to its in situ expression pattern the DP214 protein is specifically expressed in the mesenchyme of the developing pancreas, the lung, the stomach, the kidney, and specific areas of the ectoderm such as ectodermal ridge and hair follicles. Therefore, the nucleic acid and protein of the invention are useful in medical, e.g. diagnostic or therapeutic applications comprising diseases and disorders of the pancreas (such as diabetes), and related diseases and disorders (such as adipositas), of the lung, of the stomach, of the kidney, of the skin, of appendages such as limbs and hair, and others. Hence the protein of the invention is useful as a diagnostic marker or as a target for prevention or treatment of the above stated diseases and disorders.

Therapeutic uses for the invention(s) are, for example but not limited to, the following: (i) Protein therapeutic, (ii) small molecule drug target, (iii) antibody target (therapeutic, diagnostic, drug targeting/cytotoxic antibody), (iv) diagnostic and/or prognostic marker, (v) gene therapy (gene delivery/gene ablation), (vi) research tools, and (vii) tissue regeneration in vitro and in vivo (regeneration for all these tissues and cell types composing these tissues and cell types derived from these tissues.

The nucleic acids and proteins of the invention are useful in therapeutic applications implicated in various diseases and disorders as described above and/or other pathologies and disorders. For example, but not limited to, a cDNA encoding the DP214-like protein of the invention may be useful in gene therapy, and the DP214-like protein of the invention may be useful when administered to a subject in need thereof. By way of non-limiting example, the compositions of the present invention will have efficacy for prevention or treatment of patients suffering from the above mentioned diseases and disorders.

The nucleic acid encoding the DP214-like protein of the invention, or fragments thereof, is further useful in diagnostic applications, wherein the presence or amount of the nucleic acid or the protein are to be assessed. These materials are further useful in the generation of antibodies that bind immunospecifically to DP214-like proteins for use in therapeutic or diagnostic methods.

In other embodiments of the invention, pDP214 or fragments thereof may be used for therapeutic purposes. Based on the basis of in situ hybridization experiments and RT-PCR analysis results (FIGS. 4, 5, and 6), pDP214 is believed to function in the pancreas, for example in pancreatic islet cells, in crypt cells of the intestine, and in adipose tissue.

For example, in one aspect, antibodies which are specific for pDP214 may be used directly as an antagonist, or indirectly as a targeting or delivery mechanism for bringing a pharmaceutical agent to cells or tissue which express pDP214. The antibodies may be generated using methods that are well known in the art. Such antibodies may include, but are not limited to, polyclonal, monoclonal, chimeric, single chain, Fab fragments, and fragments produced by a Fab expression library. Neutralizing antibodies, (i.e., those which inhibit dimer formation) are especially preferred for therapeutic use.

For the production of antibodies, various hosts including goats, rabbits, rats, mice, humans, and others, may be immunized by injection with pDP214 or any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, keyhole limpet hemocyanin, and dinitrophenol. Among adjuvants used in human, BCG (Bacille Calmette-Guerin) and Corynebacterium parvum are especially preferable.

It is preferred that the peptides, fragments, or oligopeptides used to induce antibodies to pDP214 have an amino acid sequence consisting of at least five amino acids, and more preferably at least 10 amino acids. It is preferable that they are identical to a portion of the amino acid sequence of the natural protein, and they may contain the entire amino acid sequence of a small, naturally occurring molecule. Short stretches of pDP214 amino acids may be fused with those of another protein such as keyhole limpet hemocyanin and antibody produced against the chimeric molecule.

Monoclonal antibodies to pDP214 may be prepared using any technique which provides for the production of antibody molecules by continuous cell lines in culture. These include, but are not limited to, the hybridoma technique, the human B-cell hybridoma technique, and the EBV-hybridoma technique (Köhler, G. et al. (1975) Nature 256:495-497; Kozbor, D. et al. (1985) J. Immunol. Methods 81:31-42; Cote, R. J. et al. (Proc. Natl. Acad. Sci. 80:2026-2030; Cole, S. P. et al. (1984) Mol. Cell Biol. 62:109-120 In addition, techniques developed for the production of “chimeric antibodies”, the splicing of mouse antibody genes to human antibody genes to obtain a molecule with appropriate antigen specificity and biological activity can be used (Morrison, S. L. et al. (1984) Proc. Natl. Acad. Sci. 81:6851-6855; Neuberger, M. S. et al. (1984) Nature 312:604-608; Takeda, S. et al. (1985) Nature 314:452-454). Alternatively, techniques described for the production of single chain antibodies may be adapted, using methods known in the art, to produce pDP214-specific single chain antibodies. Antibodies with related specificity, but of distinct idiotypic composition, may be generated by chain shuffling from random combinatorial immunoglobulin libraries (Burton, D. R. (1991) Proc. Natl. Acad. Sci. 88:111 20-3). Antibodies may also be producing by inducing in vivo production in the lymphocyte population or by screening recombinant immunoglobulin libraries or panels of highly specific binding reagents as disclosed in the literature (Orlandi, R. et al. (1989) Proc. Natl. Acad. Sci. 86:3833-3837; Winter, G. et al. (1991) Nature 349:293-299).

Antibody fragments which contain specific binding sites for pDP214 may also be generated. For example, such fragments include, but are not limited to, the F(ab′)₂ fragments which can be produced by pepsin digestion of the antibody molecule and the Fab fragments which can be generated by reducing the disulfide bridges of F(ab′)₂ fragments. Alternatively, Fab expression libraries may be constructed to allow rapid and easy identification of monoclonal Fab fragments with the desired specificity (Huse, W. D. et al. (1989) Science 254:1275-1281).

Various immunoassays may be used for screening to identify antibodies having the desired specificity. Numerous protocols for competitive binding and immunoradiometric assays using either polyclonal or monoclonal antibodies with established specificities are well known in the art. Such immunoassays typically involve the measurement of complex formation between pDP214 and its specific antibody. A two-site; monoclonal-based immunoassay utilizing monoclonal antibodies reactive to two non-interfering pDP214 epitopes is preferred, but a competitive binding assay may also be employed (Maddox, supra).

In another embodiment of the invention, the polynucleotides encoding pDP214, or any fragment thereof, or antisense molecules, may be used for therapeutic purposes. In one aspect, antisense to the polynucleotide encoding pDP214 may be used in situations in which it would be desirable to block the transcription of the mRNA. In particular, cells may be transformed with sequences complementary to polynucleotides encoding pDP214. Thus, antisense molecules may be used to modulate pDP214 activity, or to achieve regulation of gene function. Such technology is now well know in the art, and sense or antisense oligomers or larger fragments, can be designed from various locations along the coding or control regions of sequences encoding pDP214. Expression vectors derived from retroviruses, adenovirus, herpes or vaccinia viruses, or from various bacterial plasmids may be used for delivery of nucleotide sequences to the targeted organ, tissue or cell population. Methods which are well known to those skilled in the art can be used to construct recombinant vectors which will express antisense molecules complementary to the polynucleotides of the gene encoding pDP214. These techniques are described both in Sambrook et. al. (supra) and in Ausubel et. al. (supra). Genes encoding pDP214 can be turned off by transforming a cell or tissue with expression vectors which express high levels of a polynucleotide or fragment thereof which encodes pDP214. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector and even longer if appropriate replication elements are part of the vector system.

As mentioned above, modifications of gene expression can be obtained by designing antisense molecules, DNA, RNA, or PNA, to the control regions of the gene encoding pDP214, i.e., the promoters, enhancers, and introns. Oligonucleotides derived from the transcription initiation site, e.g., between positions −10 and +10 from the start site, are preferred. Similarly, inhibition can be achieved using “triple helix” base-pairing methodology. Triple helix pairing is useful because it cause inhibition of the ability of the double helix to open sufficiently for the binding of polymerases, transcription factors, or regulatory molecules. Recent therapeutic advances using triplex DNA have been described in'the literature (Gee, J. E. et al. (1994) In; Huber, B. E. and B. I. Carr, Molecular and Immunologic Approaches, Futura Publishing Co., Mt. Kisco, N.Y.). The antisense molecules may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. Examples which may be used include engineered hammerhead motif ribozyme molecules that can be specifically and efficiently catalyze endonucleolytic cleavage of sequences encoding pDP214. Specific ribozyme cleavage sites within any potential RNA target are initially identified by scanning the target molecule for ribozyme cleavage sites which include the following sequences: GUA, GUU, and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Antisense molecules and ribozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding pDP214. Such DNA sequences may be incorporated into a variety of vectors with suitable RNA polymerase promoters such as T7 or SP6. Alternatively, these cDNA constructs that synthesize antisense RNA constitutively or inducibly can be introduced into cell lines, cells, or tissues. RNA molecules may be modified to increase intracellular stability and half-life. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases. Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection and by liposome injections may be achieved using methods which are well known in the art. Any of the therapeutic methods described above may be applied to any suitable subject including, for example, mammals such as dogs, cats, cows, horses, rabbits, monkeys, and most preferably, humans.

An additional embodiment of the invention relates to the administration of a pharmaceutical composition, in conjunction with a pharmaceutically acceptable carrier, for any of the therapeutic effects discussed above. Such pharmaceutical compositions may comprise DP214 nucleic acids, DP214 proteins, antibodies to pDP214, mimetics, agonists, antagonists, or inhibitors of pDP214 as an active agent. The compositions may be administered alone or in combination with at least one other agent, such as stabilizing compound, which may be administered in any sterile, biocompatible pharmaceutical carrier, including, but not limited to, saline, buffered saline, dextrose, and water. The compositions may be administered to a patient alone, or in combination with other agents, drugs or hormones. The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient. Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxy propylmethyl-cellulose, or sodium carboxymethylcellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate. Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coating for product identification or to characterize the quantity of active compound, i.e., dosage. Pharmaceutical preparations which can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions. For topical or nasal administration, penetrants appropriate to the particular barrier to be permeated are used in the formulation. Such penetrants are generally known in the art.

The pharmaceutical compositions of the present invention may be manufactured in a manner that is known in the art, e.g., by means-of conventional mixing, dissolving, granulating, dragee-making, levigating, emulsifying, encapsulating, entrapping, or lyophilizing processes. The pharmaceutical composition may be provided as a salt and can be formed with many acids, including but not limited to, hydrochloric, sulfuric; acetic, lactic, tartaric, malic, succinic, etc. Salts tend to be more soluble in aqueous or other protonic solvents than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, that is combined with buffer prior to use. After pharmaceutical compositions have been prepared, they can be placed in an appropriate container and labeled for treatment of an indicated condition. For administration of pDP214, such labeling would include amount, frequency, and method of administration.

Pharmaceutical compositions suitable for use in the invention include compositions wherein the active ingredients are contained in an effective amount to achieve the intended purpose. The determination of an effective dose is well within the capability of those skilled in the art. For any compounds, the therapeutically effective does can be estimated initially either in cell culture assays, e.g., of preadipoctic cell lines, or in animal models, usually mice, rabbits, dogs, or pigs. The animal model may also be used to determine the appropriate concentration range and route of administration. Such information can then be used to determine useful doses and routes for administration in humans. A therapeutically effective dose refers to that amount of active ingredient, for example pDP214 or fragments thereof, antibodies of pDP214, condition. Therapeutic efficacy can toxicity may be determined by standard pharmaceutical procedures in cell cultures or experimental animals, e.g., ED50 (the does therapeutically effective in 50% of the population) and LD50 (the dose lethal to 50% of the population). The dose ratio between therapeutic and toxic effects is the therapeutic index, and it can be expressed as the ratio, LD50/ED50. Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage from employed, sensitivity of the patient, and the route of administration. The exact dosage will be determined by the practitioner, in light of factors related to the subject that requires treatment. Dosage and administration are adjusted to provide sufficient levels of the active moiety or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation. Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art employ different formulations for nucleotides than for proteins or their inhibitors. Similarly, delivery of polynucleotides or polypeptides will be specific to particular cells, conditions, locations, etc.

In another embodiment, antibodies which specifically bind pDP214 may be used for the diagnosis of conditions or diseases characterized by expression of pDP214, or in assays to monitor patient's being treated with pDP214, agonists, antagonists or inhibitors. The antibodies useful for diagnostic purposes may be prepared in the same manner as those described above for therapeutics. Diagnostic assays for pDP214 include methods which utilize the antibody and a label to detect pDP214 in human body fluids or extracts of cells or tissues. The antibodies may be used with or without modification, and may be labeled by joining them, either covalently or non-covalently, with a reporter molecule. A wide variety of reporter molecules which are known in the art may be used several of which are described above.

A variety of protocols including ELISA, RIA, and FACS for measuring pDP214 are known in the art and provide a basis for diagnosing altered or abnormal levels of pDP214 expression. Normal or standard values for pDP214 expression are established by combining body fluids or cell extracts taken from normal mammalian subjects, preferably human, with antibody to pDP214 under conditions suitable for complex formation. The amount of standard complex formation may be quantified by various methods, but preferably by photometric, means. Quantities of pDP214 expressed in control and disease, samples from biopsied tissues are compared with the standard values. Deviation between standard and subject values establishes the parameters for diagnosing disease.

In another embodiment of the invention, the polynucleotides encoding pDP214 may be used for diagnostic purposes. The polynucleotides which may be used include oligonucleotide sequences, antisense RNA and DNA molecules, and PNAs. The polynucleotides may be used to detect and quantitate gene expression in biopsied tissues in which expression of pDP214 may be correlated with disease. The diagnostic assay may be used to distinguish between absence, presence, and excess expression of pDP214, and to monitor regulation of pDP214 levels during therapeutic intervention.

In one aspect, hybridization with PCR probes which are capable of detecting polynucleotide sequences, including genomic sequences, encoding pDP214 or closely related molecules, may be used to identify nucleic acid sequences which encode pDP214. The specificity of the probe, whether it is made from a highly specific region, or a less specific region, and the stringency of the hybridization or amplification (maximal, high, intermediate, or low) will determine whether the probe identifies only naturally occurring sequences encoding pDP214, alleles, or related sequences. Probes may also be used for the detection of related sequences, and should preferably contain at least 50% of the nucleotides from any of the pDP214 encoding sequences. The hybridization probes of the subject invention may be DNA or RNA and derived from the nucleotide sequence of SEQ ID NO:1 or from genomic sequences or from human homolog sequences including promoter, enhancer elements, and introns of the naturally occurring pDP214. Means for producing specific hybridization probes for DNAs encoding pDP214 include the cloning of nucleic acid sequences encoding pDP214 or pDP214 derivatives into vectors for the production of mRNA probes. Such vectors are known in the art, commercially available, and may be used to synthesize RNA probes in vitro by means of the addition of the appropriate RNA polymerases and the appropriate labeled nucleotides. Hybridization probes may be labeled by a variety of reporter groups, for example, radionuclides such as 32P or 35S, or enzymatic labels, such as alkaline phosphatase coupled to the probe via avidin/biotin coupling systems, and the like. Polynucleotide sequences encoding pDP214 may be used for the diagnosis of conditions or diseases which are associated with expression of pDP214. Examples of such conditions or diseases are as describe above.

Polynucleotide sequences encoding pDP214 may also be used to monitor the progress of patients receiving treatment for diseases and disorders, for example, but not limited to, of the pancreas (such as diabetes), and related diseases and disorders (such as adipositas), of the lung, of the stomach, of the kidney, and others.

The polynucleotide sequences encoding pDP214 may be used in Southern or Northern analysis, dot blot, or other membrane-based technologies; in PCR technologies; or in dip stick, pin, ELISA or chip assays utilizing fluids or tissues from patient biopsies to detect altered pDP214 expression. Such qualitative or quantitative methods are well known in the art. In a particular aspect, the nucleotide sequences encoding pDP214 may be useful in assays that detect activation or induction of various diseases and disorders, for example, but not limited to, of the pancreas (such as diabetes), and related diseases and disorders (such as adipositas), of the lung, of the kidney, and others.

The nucleotide sequences encoding pDP214 may be labeled by standard methods, and added to a fluid or tissue sample from a patient under conditions suitable for the formation of hybridization complexes. After a suitable incubation period, the sample is washed and the signal is quantitated and compared with a standard value. If the amount of signal in the biopsied or extracted sample is significantly altered from that of a comparable have hybridized with nucleotide sequences in the sample, and the presence of altered levels of nucleotide sequences encoding pDP214 in the sample indicates the presence of the associated disease. Such assays may also be used to evaluate the efficacy of a particular therapeutic treatment regimen in animal studies, in clinical trials, or in monitoring the treatment of an individual patient. In order to provide a basis for the diagnosis of disease associated with expression of pDP214, a normal or standard profile for expression is established. This may be accomplished by combining body fluids or cell extracts taken from normal subjects, either animal or human, with a sequence, or a fragment thereof, which encodes pDP214, under conditions suitable for hybridization or amplification. Standard hybridization may be quantified by comparing the values obtained from normal subjects with those from an experiment where a known amount of a substantially purified polynucleotide is used. Standard values obtained from normal samples may be compared with values obtained from samples from patients who are symptomatic for disease. Deviation between standard and subject values is used to establish the presence of disease. Once disease is established and a treatment protocol is initiated, hybridization assays may be repeated on a regular basis to evaluate whether the level of expression in the patient begins to approximate that which is observed in the normal patient. The results obtained from successive assays may be used to show the efficacy of treatment over a period ranging from several days to months.

With respect to diseases and disorders, for example, but not limited to, of the pancreas (such as diabetes), and related diseases and disorders (such as adipositas), of the lung, of the stomach, of the kidney, and others. The presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow to employ preventative measures or aggressive treatment earlier thereby preventing the development or further progression of the pancreatic diseases and disorders.

Additional diagnostic uses for oligonucleotides (oligomers) designed from the sequences encoding pDP214 may involve the use of amplification reactions such as PCR. Such oligomers may be chemically synthesized, generated enzymatically, or produced from a recombinant source. Oligomers will preferably consist of two nucleotide sequences, one with sense orientation (5′.fwdarw.3′) and another with antisense (3′.rarw.5′), employed under optimized conditions for identification of a specific gene or condition. The same two oligomers, nested sets of oligomers, or even a degenerate pool of oligomers may be employed under less stringent conditions for detection and/or quantitation of closely related DNA or RNA sequences. Methods which may also be used to quantitate the expression of pDP214 include radiolabeling or biotinylating nucleotides, coamplification of a control nucleic acid, and standard curves onto which the experimental results are interpolated (Melby, P. C. et al. (1993) J. Immunol. Methods, 159:235-244; Duplaa, C. et al. (1993) Anal. Biochem. 212:229-236. The speed of quantitation of multiple samples may be accelerated by running the assay in an ELISA format where the oligomer of interest is presented in various dilutions and a spectrophotometric or colorimetric response gives rapid quantitation.

In another embodiment of the invention, the nucleic acid sequences which encode pDP214 may also be used to generate hybridization probes which are useful for mapping the naturally occurring genomic sequence. The sequences may be mapped to a particular chromosome or to a specific region of the chromosome using well known techniques. Such techniques include FISH, FACS, or artificial chromosome constructions, such as yeast artificial chromosomes, bacterial artificial chromosomes, bacterial P1 constructs or single chromosome cDNA libraries as reviewed in Price, C. M. (1993) Blood Rev. 7:127-134, and Trask, B. J. (1991) Trends Genet. 7:149-154. FISH (as described in Verma et al. (1988), Human Chromosomes: A Manual of Basic Techniques, Pergamon Press, New York, N.Y.) may be correlated with other physical chromosome mapping techniques and genetic map data. Examples of genetic map data can be found in the 1994 Genome Issue of Science (265:1981 f). Correlation between the location of the gene encoding pDP214 on a physical chromosomal map and a specific disease, or predisposition to a specific disease, may help delimit the region of DNA associated with that genetic disease.

The nucleotide sequences of the subject invention may be used to detect differences in gene sequences between normal, carrier, or affected individuals. In situ hybridization of chromosomal preparations and physical mapping techniques such as linkage analysis using established chromosomal markers may be used for extending genetic maps. Often the placement of a gene on the chromosome of another mammalian species, such as mouse, may reveal associated markers even if the number or arm of a particular human chromosome is not known. New sequences can be assigned to chromosomal arms, or parts thereof, by physical mapping. This provides valuable information to investigators searching for disease genes using positional cloning or other gene discovery techniques. Once the disease or syndrome has been crudely localized by genetic linkage to a particular genomic region, for example, AT to 11q22-23 (Gatti, R. A. et al. (1988) Nature 336:577-580), any sequences mapping to that area may represent associated or regulatory genes for further investigation. The nucleotide sequence of the subject invention may also be used to detect differences in the chromosomal location due to translocation, inversion, etc. among normal, carrier, or affected individuals.

In another embodiment of the invention, pDP214, its catalytic or immunogenic fragments or oligopeptides thereof, can be used for screening libraries of compounds in any of a variety of drug screening techniques. The fragment employed in such screening may be free in solution, affixed to a solid support, borne on a cell surface, or located intracellularly. The formation of binding complexes, between pDP214 and the agent tested, may be measured.

Another technique for drug screening which may be used provides for high throughput screening of compounds having suitable binding affinity to the protein of interest as described in published PCT application WO84/03564. In this method, as applied to pDP214 large numbers of different small test compounds are synthesized on a solid substrate, such as plastic pins or some other surface. The test compounds are reacted with pDP214, or fragments thereof, and washed. Bound pDP214 is then detected by methods well known in the art. Purified pDP214 can also be coated directly onto plates for use in the aforementioned drug screening techniques. Alternatively, non-neutralizing antibodies can be used to capture the peptide and immobilize it on a solid support. In another embodiment, one may use competitive drug screening assays in which neutralizing antibodies capable of binding pDP214 specifically compete with a test compound for binding pDP214. In this manner, the antibodies can be used to detect the presence of any peptide which shares one or more antigenic determinants with pDP214. In additional embodiments, the nucleotide sequences which encode pDP214, may be used in any molecular biology techniques, provided these techniques rely on properties of nucleotides that include, but are not limited to, such properties as the triplet genetic code and specific base pair interactions.

The examples below are provided to illustrate the subject invention and are not included for the purpose of limiting the invention.

### EXAMPLES

### Example 1

### DPd6 Chick cDNA Library Construction

The Chick DPd6 cDNA library was constructed from dorsal pancreatic buds dissected from 6 day old, chick embryos. The frozen tissue was homogenized and lysed using a Brinkmann POLYTRON homogenizer PT-3000 (Brinkman Instruments, Westbury, N.J.) in guanidinium isothiocyanate solution. The lysates were centrifuged over a 5.7 M CsCl cushion using as Beckman SW28 rotor in a Beckman L8-70M ultracentrifuge (Beckman Instruments, Fullerton, Calif.) for 18 hours at 25,000 rpm at ambient temperature. The RNA was extracted with acid phenol pH 4.7, precipitated using 0.3 M sodium acetate and 2.5 volumes of ethanol, resuspended in RNase free water, and DNase treated at 37° C. The RNA extraction was repeated with acid phenol pH 4.7 and precipitated with sodium acetate and ethanol as before. The mRNA was then isolated using the Micro-FastTrack 2.0 mRNA isolation kit (Invitrogen, Groningen, Netherlands) and used to construct the cDNA libraries. The mRNAs were handled according to the recommended protocols in the SUPERSCRIPT cDNA synthesis and plasmid cloning system (Cat. #18248-013. Gibco/BRL). Following transformation into DH10B host cells, single colonies were picked and the subjected to PCR in order to amplify the cloned cDNA insert. Amplified PCR fragments representing single cDNA inserts were subsequently in vitro transcribed to generate Digoxygenin labelled RNA probes (Roche). The RNA probes were used in a whole-mount in situ screen to determine the expression of their respective gene products in early chick embryos. The plasmid DP214-containing the chick DP214-like gene was identified because of its striking expression in the pancreatic epithelium.

### Example 2

### In Situ Hybridizations and RT-PCR Analysis

Whole-mount in situ hybridizations were performed according to standard protocols and as described previously (Pelton, R. W. et al., (1990) Development 110,609-620; Belo, J. A. et al., (1997) Mech. Dev. 68, 45-57). Isolation of total RNA from cell culture was carried out using TRIZOL; (Life Technologies, Karlsruhe, Germany).

### Example 3

### Isolation and Sequencing of cDNA Clones

Plasmid DNA was released from the cells and purified using the REAL PREP 96-well plasmid isolation kit (Catalog #26173, QIAGEN). This kit enabled the simultaneous purification of 96 samples in a 96-well block using multi-channel reagent dispensers. The recommended protocol was employed except for the following changes: 1) the bacteria were cultured in 1 ml of sterile Terrific Broth (Catalog #22711, LIFE TECHNOLOGIES.TM., Gaithersburg, Md.) with carbenicillin at 25 mg/L and glycerol at, 0.4%; 2) after inoculation, the cultures were incubated for 19 hours and at the, end of incubation, the cells were lysed with 0.3 ml of lysis buffer; and 3) following isopropanol precipitation, the plasmid DNA pellet was resuspended in 0.1 ml of distilled water. After the last step in the protocol, samples were transferred to a 96-well block for storage at 4° C. The cDNAs were sequenced by GATC Biotech AG (Konstanz, Germany) accoding to standard protocols known to those skilled in the art.

### Example 4

### Homology Searching of cDNA Clones and their Deduced Proteins

After the reading frame was determined, the nucleotide sequences of the Sequence Listing as well as the amino acid sequences deduced from them were used as query sequences against databases such as GenBank, SwissProt, BLOCKS, and Pima II. These databases, which contain previously identified and annotated sequences, were searched for regions of homology (similarity) using BLAST, which stands for Basic Local. Alignment Search Tool (Altschul S. F. (1993) J. Mol. Evol. 36:290-300; Altschul, S. F. et al. (1990) J. Mol. Biol. 215:403-10). BLAST produced alignments of both nucleotide and amino acid sequences to determine sequence similarity. Because of the local nature of the alignments, BLAST was especially useful in determining exact matches or in identifying homologs which may be of prokaryotic (bacterial) or eukaryotic (animal, fungal, or plant) origin. Other algorithms such as the one described in Smith et al. (1992, Protein Engineering 5:35-51), incorporated herein by reference, could have been used when dealing with primary sequence patterns and secondary structure gap penalties. The sequences disclosed in this application have lengths of at least 49 nucleotides, and no more than 12% uncalled bases (where N is recorded rather than A, C, G, or T). The BLAST approach, as detailed in Karlin et al. (supra) and incorporated herein by reference, searched for matches between a query sequence and a database sequence. BLAST evaluated the statistical significance of any matches found, and reported only those matches that satisfy the user-selected threshold of significance. In this application, threshold was set at 10-25 for nucleotides and 10-14 for peptides. Incyte nucleotide sequences were searched against the GenBank databases for primate (pri), rodent (rod), and other mammalian sequences (mam); and deduced amino acid sequences from the same clones were then searched against GenBank functional protein databases, mammalian (mamp), vertebrate (vrtp), and eukaryote (eukp) for homology. The relevant database for a particular match were reported as Glxxx±p (where xxx is pri; rod; etc.; and if present, p=peptide. The product score is calculated as follows: the % nucleotide or amino acid identity [between the query and reference sequences] in BLAST is multiplied by the % maximum possible BLAST score [based on the lengths of query and reference sequences] and then divided by 100. Where an, Incyte Clone was homologous to several sequences, up to five matches were provided with their relevant scores. In an analogy to the hybridization procedures used in the laboratory, the electronic stringency for an exact match was set at 70, and the conservative lower limit for an exact match was set at approximately 40 (with 1-2% error due to uncalled bases).

### Example 5

### Extension of pDP214-Encoding Polynucleotides to Full Length or to Recover, Regulatory Sequences

Full length pDP214-encoding nucleic acid sequence (SEC ID NO:2) is used to design oligonucleotide primers for extending a partial nucleotide sequence to full length or for obtaining 5′ or 3′, intron or other control sequences from genomic libraries. One primer is synthesized to initiate extension in the antisense direction (XLR) and the other is synthesized to extend sequence in the sense direction (XLF). Primers are used to facilitate the extension of the known sequence “outward” generating amplicons containing new, unknown nucleotide sequence fore the region of interest. The initial primers are designed from the cDNA using OLIGO 4.06 primer analysis software (National Biosciences), or another appropriate program, to be 22-30 nucleotides in length, to have a GC content of 50% or more, and to anneal to the target sequence at temperatures about 68°-72° C. Any stretch of nucleotides which would result in hairpin dimerizations is avoided. The original, selected cDNA libraries, or a human genomic-library are used to extend the sequence, the latter is most useful to obtain 5′ upstream regions. If more extension is necessary or desired, additional sets of primers are designed to further extend the known region. By following the instructions for the XL-PCR kit (Perkin Elmer) and thoroughly mixing the enzyme and reaction mix, high fidelity amplification is obtained. Beginning with 40 pmol of each primer and the recommended concentrations of all other components of the kit, PCR is performed using the Peltier thermal cycler (PTC200; M. J. Research, Watertown, Mass.) and the, following parameters:

| | |
|---|---|
| Step 1 | 94° C. for 1 min (initial denaturation) |
| Step 2 | 65° C. for 1 min |
| Step 3 | 68° C. for 6 min |
| Step 4 | 94° C. for 15 sec |
| Step 5 | 65° C. for 1 min |
| Step 6 | 68° C. for 7 min |
| Step 7 | Repeat step 4-6 for 15 additional cycles |
| Step 8 | 94° C. for 15 sec |
| Step 9 | 65° C. for 1 min |
| Step 10 | 68° C. for 7-15 min |
| Step 11 | Repeat step 8-10 for 12 cycles |
| Step 12 | 72° C. for 8 min |
| Step 13 | 4° C. (and holding) |

A 5-10 μl aliquot of the reaction mixture is analyzed by electrophoresis on a low concentration (about 0.6-0.8% agarose mini-gel to determine which reactions were successful in extending the sequence. Bands thought to contain the largest products are selected and removed from the gel. Further purification involves using a commercial gel extraction method such as the QIAQUICK DNA purification kit (QIAGEN). After recovery of the DNA, Klenow enzyme is used to trim single-stranded, nucleotide overhangs creating blunt ends which facilitate religation and cloning. After ethanol precipitation, the products are redissolved in 13 μl of ligation buffer, 1 μl T4-DNA ligase (15 units) and 1 μl T4 polynucleotide kinase are added, and the mixture is incubated at room temperature for 2-3 hours or overnight at 16° C. Competent E. coli cells (in 40 μl of appropriate media) are transformed with 3 μl of ligation mixture and cultured in 80 μl of SOC medium (Sambrook et al., supra). After incubation for one hour at 37° C., the whole transformation mixture is plated on Luria Bertani (LB)-agar (Sambrook et al., supra) containing 2×Carb. The following day, several colonies are randomly picked from each plate and cultured in 150 μl of liquid LB/2×Carb medium placed in an individual well of an appropriate, commercially-available, sterile 96-well microtiter plate. The following day, 5 μl of each overnight culture is transferred into a non-sterile 96-well plate and after dilution 1:10 with water, 5 μl of each sample is transferred into a PCR array. For PCR amplification, 18 μl of concentrated PCR reaction mix (3.3×) containing 4 units of rTth DNA polymerase, a vector primer, and one or both of the gene specific primers used for the extension reaction are added to each well. Amplification is performed using the following conditions:

| | |
|---|---|
| Step 1 | 94° C. for 60 sec |
| Step 2 | 94° C. for 20 sec |
| Step 3 | 55° C. for 30 sec |
| Step 4 | 72° C. for 90 sec |
| Step 5 | Repeat steps 2-4 for an additional 29 cycles |
| Step 6 | 72° C. for 180 sec |
| Step 7 | 4° C. (and holding) |

Aliquots of the PCR reactions are run on agarose gels together with molecular weight markers. The sizes of the PCR products are compared to the original partial cDNAs, and appropriate clones are selected, ligated into plasmid, and sequenced.

### Example 6

### Labeling and Use of Hydridization Probes

Hybridization probes derived from SEQ ID NO:2 are employed to screen cDNAs, genomic DNAs, or mRNAs. Although the labeling of oligonucleotides, consisting of about 20 base-pairs, is specifically described, essentially the same procedure is used with larger cDNA fragments. Oligonucleotides are designed using state-of-the-art software such as OLIGO 4.06 primer analysis software (National Biosciences, labeled by combining 50 pmol of each oligomer and 250 μCi of [.gamma.-32 P] adenosine triphosphate (Amersham) and T4 polynucleotide kinase (DuPont Nen®, Boston, Mass.). The labelled oligonucleotides are substantially purified with SEPHADEX G-25 superfine resin column (Pharmacia & Upjohn). A portion containing 107 counts per minute of each of the sense and antisense oligonucleotides is used in a typical membrane based hybridization analysis of human genomic DNA digested with one of the following membranes (Ase I, Bgl II, EcoRI, Pst I, Xba 1, or Pvu II; DuPont NEN®). The DNA from each digest is fractionated on a 0.7 percent agarose gel and transferred to nylon membranes (NYTRAN PLUS membrane, Schleicher & Schuell, Durham, N.H.). Hybrization is carried out for 16 hours at 40° C. To remove nonspecific signals, blots are sequentially washed at room temperature under increasingly stringent conditions up to 0.1×saline solution citrate and 0.5% sodium dodecyl sulfate. After XOMAI AR Autoradiography film (Kodak Rochester, N.Y.) is exposed to the blots, or the blots are placed in a PHOSPHOIMAGER (Molecular Dynamics, Sunnyvale, Calif.) for several hours, hybridization patterns are compared visually.

### Example 7

### Antisense Molecules

Antisense molecules to the pDP214-encoding sequence, or any part thereof, is used to inhibit in vivo or in vitro expression of naturally occurring pDP214. Although use of antisense oligonucleotides, comprising about 20 base-pairs; is specifically described, essentially the same procedure is used with larger cDNA fragments. An oligonucleotide based on the coding sequences of pDP214, as shown in FIGS. 1A, 1B and 1C, is used to inhibit expression of naturally occurring pDP214. The complementary oligonucleotide is designed from the most unique 5′ sequence as shown in FIGS. 1A, 1B and 1C and used either to inhibit transcription by preventing promoter binding to the upstream nontranslated sequence to translation of an pDP214-encoding transcript by preventing the ribosome from binding. Using an appropriate portion of the signal and 5′ sequence of SEQ ID NO:2, an effective antisense oligonucleotide includes any 15-20 nucleotides spanning the region which translates into the signal or 5′ coding sequence of the polypeptide as shown in FIGS. 1A, 1B and 1C.

### Example 8

### Expression of pDP214

Expression of pDP214 is accomplished by subcloning the cDNAs into appropriate vectors and transforming the vectors into host cells. In this case, the cloning vector, PSPORT 1, previously used for the generation of the cDNA library is used to express pDP214 in E. coli. Upstream of the cloning site, this vector contains a promoter for 62-galactosidase, followed by sequence containing the amino-terminal Met, and the subsequent seven residues of 0.9-galactosidase. Immediately following these eight residues is a bacteriophage promoter useful for transcription and a linker containing a number of unique restriction sites. Induction of an isolated, transformed bacterial strain with IPTG using standard methods produces a fusion protein which consists of the first eight residues of β-galactosidase, about 5 to 15 residues of linker, and the full length protein. The signal residues direct the secretion of pDP214 into the bacterial growth media which can be used directly in the following assay for activity.

### Example 9

### Production of pDP214 Specific Antibodies

pDP214 that is substantially purified using PAGE electrophoresis (Sambrook, supra), or other purification techniques, is used to immunize rabbits and to produce antibodies using standard protocols. The amino acid sequence deduced from SEQ ID NO:2 is analyzed using DNASTAR software (DNASTAR Inc) to determine regions of high immunogenicity and a corresponding oligopolypeptide is synthesized and used to raise antibodies by means known to those of skill in the art. Selection of appropriate epitopes, such as those near the C-terminus or in hydrophilic regions, is described by Ausubel et al. (supra), and others.

Typically, the oligopeptides are 15 residues in length, synthesized using an Applied Biosystems 431A peptide synthesizer 431A using fmoc-chemistry, and coupled to keyhole limpet hemocyanin (KLH, Sigma, St. Louis, Mo.) by reaction with N-maleimidobenzoyl-N-hydroxysuccinimide ester (MBS; Ausubel et al., supra). Rabbits are immunized with the oligopeptide-KLH complex in complete Freund's adjuvant. The resulting antisera are tested for antipeptide activity, for example, by binding the peptide to plastic, blocking with 1% BSA, reacting with rabbit antisera, washing, and reacting with radioiodinated, goat anti-rabbit IgG.

### Example 10

### Identification of Molecules which Interact with pDP214

pDP214 or biologically active fragments thereof are labeled with 125 I Bolton-Hunter reagent (Bolton et al. (1973) Biochem. J. 133:529). Candidate molecules previously arrayed in the wells of a multi-well plate are incubated with the labeled pDP214, washed and any wells with labeled pDP214 complex are assayed. Data obtained using different concentrations of pDP214 are used to calculate values for the number, affinity, and association of pDP214 with the candidate molecules. All publications and patents mentioned in the above specification are herein incorporated by reference.

Various modifications and variations of the described method and system of the invention will be apparent to those, skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A pharmaceutical composition comprising as an active agent a nucleic acid molecule of the DP214 gene family or a polypeptide encoded thereby or a fragment or variant of said nucleic acid molecule or said polypeptide or an antibody, aptamer or other receptor recognizing a nucleic acid molecule of the DP214 gene family or a polypeptide encoded thereby.

2. The composition of claim 1 wherein the nucleic acid is a chicken DP214 nucleic acid or a human homolog or a fragment or a variant thereof.

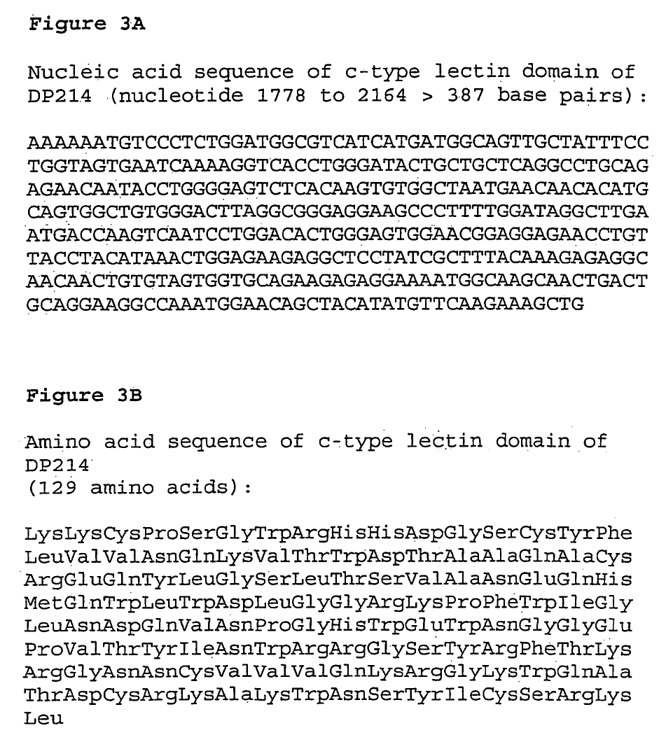

3. The composition of claim 1 or 2 wherein the nucleic acid molecule is a C-type lectin domain comprising the sequence which is designated as CLT1 in FIG. 3 or a human homolog or a fragment or a variant thereof.

4. The composition of any one of claims 1-3, wherein the nucleic acid molecule encodes a polypeptide expressed in specific tissues, wherein said nucleic acid molecule
(a) hybridizes at 55° C. in a solution containing 1×SSC and 0.1% SDS to a nucleic acid molecule encoding the amino acid sequence of SEQ ID NO: 1 or the complementary strand thereof;
(b) it is degenerate with respect to the nucleic acid molecule of (a)
(c) encodes a polypeptide which is at least 85%, preferably at least 90%, more preferably at least 95%, more preferably at least 98% and up to 99.6% identical to SEQ ID NO: 2, or
(d) differs from the nucleic acid molecule of (a) to (c) by mutation and wherein said mutation causes an alteration, deletion, duplication or premature stop in the encoded Polypeptide.

5. The composition of any one of claims 1-4 wherein the nucleic acid molecule is a DNA molecule, particularly a cDNA or a genomic DNA.

6. The composition of any one of claims 1-5 wherein the nucleic acid molecule is associated with differentiation or developmental control, particularly associated with the development of the pancreas, more particularly causally associated with the development of the pancreas in mammals.

7. The composition of any one of claims 1-6 wherein the nucleic acid molecule encodes a protein that is expressed in the mesenchyme of the developing pancreas, in the lung, the stomach, the kidney or/and specific areas of the ectoderm such as ectodermal ridge and hair follicles.

8. The composition of any one of claims 1-7 wherein the nucleic acid molecule is a recombinant or synthetic and optionally modified nucleic acid molecule.

9. The composition of claim 8 wherein the recombinant nucleic acid molecule is a vector, particularly an expression vector.

10. The composition of claim 1 wherein the active ingredient is an antibody, fragment or derivative thereof or an aptamer or another receptor specifically recognizing a nucleic acid molecule of the DP214 family or a polypeptide encoded thereby.

11. The composition of any one of claims 1-10 wherein said polypeptide is a recombinant polypeptide.

12. The composition of claim 11 wherein said polypeptide is a fusion polypeptide.

13. The composition of any one of claims 1-10 wherein said nucleic acid molecule is selected from hybridization probes, primers and antisense oligonucleotides.

14. The composition of any one of claims 1-13 for diagnostic applications.

15. The composition of any one of claims 1-13 for therapeutic applications.

16. The composition of any one of claims 1-15 for the manufacture of an agent for the diagnosis, monitoring, prevention or treatment of diseases or disorders of the pancreas, lung, stomach, kidney, skin, and/or appendages particularly in a mammal.

17. The composition of claim 16 wherein the disease or disorder is a pancreatic disease or disorder including metabolic disorders associated with an endocrine dysfunction such as diabetes or adipositas.

18. A non-human animal expressing the polypeptide of claim 11 or the fusion protein of claim 12, transfected with the vector of claim 9, or comprising the nucleic acid molecule of any one of claims 1 to 8.

19. The animal of claim 18 wherein the expression of the DP214 polypeptide is modified, e.g. increased or reduced.

20. A recombinant host cell exhibiting a modified, e.g. increased or reduced expression of a DP214 polypeptide.

21. The cell of claim 20 which is a human cell.

22. Use of the nucleic acid molecule of any one of claims 1 to 8, the vector of claim 9, the host or host cell of any one of claims 18-21, the polypeptide of claim 11, the fusion protein of claim 12, the antibody, fragment or derivative thereof, aptamer or receptor of claim 10 or the anti-sense oligonucleotide of claim 13 for controlling the function of a gene and/or a gene product which is influenced and/or modified by a DP214 polypeptide.

23. Use of the composition of any one of claims 1-17, for the manufacture of an agent for detecting and/or verifying, for the treatment, alleviation and/or prevention of diseases or disorders of the pancreas (such as diabetes), and related diseases and disorders (such as adipositas), of the lung, of the stomach, of the kidney, of the skin and of appendages and others in cells, cell masses, organs and/or subjects.

24. Use of the nucleic acid molecule of any one of claims 1 to 8, the vector of claim 9, the host or host cell of any one of claims 18-21, the polypeptide of claim 11, the fusion protein of claim 12, the antibody, fragment or derivative thereof, aptamer or receptor of claim 10 or the anti-sense oligonucleotide of claim 13 for identifying substances capable of interacting with a DP214 polypeptide.

25. A method of identifying a polypeptide involved in diseases and disorders, for example, but not limited to, of the pancreas (such as diabetes), and related diseases and disorders (such as adipositas), of the pancreas, of the lung, of the stomach, of the kidney, of the skin or of appendages, and others in a mammal comprising the steps of
(a) contacting a collection of (poly)peptides with a DP214 polypeptide or a fragment thereof under conditions that allow binding of said (poly)peptides;
(b) removing (poly)peptides from said collection of (poly)peptides that do not bind in step (a); and
(c) identifying (poly)peptides that bind to said DP214 polypeptide or the fragment thereof.

26. A method of screening for an agent which modulates the interaction of a DP214 polypeptide with a binding target/agent, comprising the steps of
(a) incubating a mixture comprising
(aa) a DP214 polypeptide, or a fragment thereof;
(ab) a binding target/agent of said DP214 polypeptide or fragment thereof; and
(ac) a candidate agent
under conditions whereby said polypeptide or fragment thereof specifically binds to said binding target/agent at a reference affinity;
(b) detecting the binding affinity of said DP214 polypeptide or fragment thereof to said binding target to determine an (candidate) agent-biased affinity; and
(c) determining a difference between (candidate) agent-biased affinity and the reference affinity.

27. A method of producing a composition comprising the (poly)peptide identified by the method of claim 25 or the agent identified by the method of claim 26 with a pharmaceutically acceptable carrier and/or diluent.

28. The method of claim 27 wherein said composition is a pharmaceutical composition for preventing, alleviating or treating diseases and disorders (such as adipositas), of the lung, of the stomach, of the kidney, of the skin and of appendages and others in cells, cell masses, organs and/or subjects, and other diseases and disorders, particularly as identified in claim 25.

29. Use of a (poly)peptide as identified in claim 25 or of an agent as identified by the method of claim 26 for the preparation of a pharmaceutical composition for the treatment, alleviation and/or prevention of diseases or disorders of the pancreas (such as diabetes), and related diseases and disorders (such as adipositas), of the lung, of the stomach, of the kidney, of the skin and of appendages and others in cells, cell masses, organs and/or subjects.

30. Use of a nucleic acid molecule as depicted in SEQ ID, NO: 1 or a homolog or a a fragment thereof for the preparation of a transgenic non-human animal which over- or underexpresses the corresponding gene product or a fragment thereof.

31. Kit comprising at least one of
(a) a nucleic acid molecule of any one of claims 1 to 8;
(b) a vector of claim 9;
(c) a host or host cell of any one of claims 18-21;
(d) a polypeptide of claim 11;
(e) a fusion protein of claim 12;
(f) an antibody or a fragment or derivative thereof or an aritiserum, an aptamer or a receptor of claim 10; and
(g) an anti-sense oligonucleotide of claim 13.
